(19) United States
(12) Patent Application Publication
(10) Pub. No.: US 2023/0366004 A1
(43) Pub. Date: Nov. 16, 2023

(54) TRAIN COMPARTMENT AIR ADJUSTMENT AND CONTROL METHOD AND APPARATUS, STORAGE MEDIUM, AND PROGRAM PRODUCT

(71) Applicant: CENTRAL SOUTH UNIVERSITY, Changsha, Hunan (CN)

(72) Inventors: Hui Liu, Changsha, Hunan (CN); Yanfei Li, Changsha, Hunan (CN); Jiahao Xie, Changsha, Hunan (CN); Jie Zhang, Changsha, Hunan (CN); Xirui Chen, Changsha, Hunan (CN)

(73) Assignee: CENTRAL SOUTH UNIVERSITY, Changsha, Hunan (CN)

(21) Appl. No.: 18/267,450

(22) PCT Filed: Oct. 9, 2021
(86) PCT No.: PCT/CN2021/122732

§ 371 Date: Jun. 14, 2023

(30) Foreign Application Priority Data
Dec. 30, 2020 (CN) 202011616109.X

(51) Int. Cl. C12Q 1/06; G01N 15/06; B60H 1/00

(52) U.S. Cl. CPC C12Q 1/06; G01N 15/06; B60H 1/008

(57) ABSTRACT

Disclosed are a train compartment air adjustment and control method and apparatus, and a storage medium and a program product. A ventilation system is adjusted according to microbial diffusion situations among various test points, so as to reduce a microbial pollution index of an area where passengers are located. The method has a guide effect on railway train air quality adjustment and control. By means of the present invention, a mapping relationship between microbial pollution and the concentration of atmospheric pollutants is studied, the problem of the real-time performance of microbial detection can be effectively solved, and the real-time adjustment and control of microbial pollution in a train compartment are guaranteed.

### FIELD OF INVENTION

The present invention relates to the field of train environment monitoring, in particular to a train compartment air adjustment and control method and apparatus, a storage medium, and a program product.

### BACKGROUND OF THE INVENTION

With continuous development of China's rail transit industry, comfort requirements of passenger trains have gradually been concerned by the public. As affected by the air pressure wave, proper pressure difference between inside and outside of a train compartment is required during high-speed running, so a high-speed train usually has a sealed body structure, and all windows cannot be opened. In this case, treatment of air pollutants inside the compartment entirely depends on a ventilation system. Therefore, quality and adjustment strategies of the ventilation system will directly affect passenger comfort. Thus, how to monitor a train environment and correspondingly adjust the ventilation system has become an urgent problem to be solved.

The prior art for environment control of train compartments mainly involves the following two aspects:
1. Installation of a novel air quality detection apparatus and a purification apparatus is proposed. For example, patent application with Publication No. CN101885338A provides an intelligent sampling, detecting and air purification apparatus for a train air conditioning and ventilation system, including a variable frequency cyclone dust catcher, a high efficiency filter, and the like. Patent application with Publication No. CN105172818A provides a special air purifier for a train, including an upper box body and a lower box body matched with the upper box body.
2. A compartment environment adjustment method based on air pollutant detection is proposed. Patent application with Publication No. CN110239577A provides a system and method for protecting train crew members' health in an internal contamination environment, the system including a basic data acquisition module, a train external air quality prediction module, a train internal air quality prediction module, and a ventilation strategy formulation module. Patent application with Publication No. CN104608785A provides an intelligent management and control method for an air conditioning system of a high-speed train.

The above methods mainly use the concentration of air pollutants such as PM_{2.5} inside a train as the basis for air quality evaluation. However, the prior art does not concern harm of biological contamination in an air environment to human health. At present, no relevant research focuses on biological pollutants in a closed environment of a train compartment. Moreover, because a measurement mechanism for microorganisms is different from that for pollutants such as PM_{2.5}, microbial measurement requires long-term colony culture, while direct detection and real-time adjustment and control of microbial are difficult.

### SUMMARY OF THE INVENTION

The technical problem to be solved by the present invention is, aiming at the deficiencies of the prior art, to provide a train compartment air adjustment and control method and apparatus, a storage medium, and a program product, where optimal-level protection measures are implemented for passenger health according to microbial distribution in a compartment.

In order to solve the foregoing technical problem, the technical solution employed by the present invention is as follows: a train compartment air adjustment and control method, wherein the method including the following steps:
1) detecting PM_{2.5} concentration, PM₁₀ concentration, CO concentration, NO₂ concentration, SO₂ concentration, O₃ concentration, and the total number of bacterial colonies at an air supply port, an air exhaust port and a seat of a train;
2) establishing, according to the PM_{2.5} concentration, PM₁₀ concentration, CO concentration, NO₂ concentration, SO₂ concentration, O₃ concentration, and the total number of bacterial colonies at each detection point in a compartment, a mapping relationship between the total number of bacterial colonies D and the concentration of air pollutants d in each micro environmental unit, where the micro environmental unit the detection point;
3) selecting a measured air pollutant concentration data set with a time length of N minutes, calculating the total number of bacterial colonies according to the mapping relationship, denoting a time series of the total number of bacterial colonies at the i^{th} seat as X_{N}ⁱ, denoting a time series of the total number of bacterial colonies at the j^{th} air supply port or air exhaust port as Y_{N}^{j}, performing; hypothesis test by using Granger causality test to determine whether there is causality between X_{N}ⁱ and Y_{N}^{j}, and then obtaining a test result set of m air supply ports and n air exhaust ports at each seat detection point;
4) obtaining a nonlinear description remodel base of all seat detection points according to the mapping relationship and the test result set; and
5) inputting ventilation rates of all air supply ports and all air exhaust ports of the train to a grey wolf optimizer, calculating fitting results of the total number of bacterial colonies at the air supply ports/air exhaust ports under different ventilation rates, inputting the fitting results to the nonlinear description model base to obtain a fitting result of the total number of bacterial colonies at each seat, and determining the ventilation rates of all the air supply ports and all the air exhaust ports by using the fitting result of the total number of bacterial colonies at each seat.

According to the present invention, the mapping relationship between microbial contamination and air pollutant concentration in the compartment is studied, and optimal protective measures for passenger health are implemented in real time according to microbial distribution in the compartment. Microorganisms in the train compartment are detected, analyzed and treated, and the ventilation system s adjusted according to the microbial distribution among the detection points; thereby reducing microbial contamination in a passenger area. The method has a guide effect on air quality adjustment and control of a railway train.

In step 2), a specific implementation process of establishing a mapping relationship between the total number of bacterial colonies D and the concentration of air pollutants d in each micro environmental unit includes:
A, reading an index data set of air pollutant concentration and total number of bacterial colonies of the current micro environmental unit at M consecutive historical moments, and dividing the index data set into a training set and a test set;
B, constructing a microorganism-air pollutant model by using a deep belief network, and training the deep belief network by using the air pollutant concentration and the total number of bacterial colonies at the same moment respectively as input and output of the deep belief network;
C, using the test set as input of the trained deep belief network, and selecting a group of parameters with highest description accuracy on the test set as a microorganism-air pollutant mapping model of the micro environmental unit; and
D, repeating steps A-C for all the micro environmental units to obtain mapping relationships between the total number of bacterial colonies and the air pollutants of m+n+p detection points, where m, n, and p are numbers of detection points at the air supply ports, the air exhaust ports, and the seats respectively.

The present invention studies the mapping relationship between microbial contamination and air pollutant concentration, which can effectively solve a real-time problem of microbial detection and ensure real-time adjustment and control on microbial contamination in train compartments.

In step 3), the test result set is φᵢ={T_{i,1}ⁱⁿ, T_{i,2}ⁱⁿ, . . . , T_{i,m}ⁱⁿ, T_{i,1}^{out}, T_{i,2}^{out}, . . . , T_{i,n}^{out}}, where T_{i,j}ⁱⁿ is a test result of the air supply port T_{i,j}ⁱⁿ=GCT(X_{N}ⁱ, Y_{N}^{j}), T_{i,j}^{out} is a test result of the air exhaust port, and T_{i,j}^{out}=GCT(X_{N}ⁱ, Y_{N}^{j}); and value of the test result T_{i,j}^{in/out} (i.e., T_{i,j}ⁱⁿ and T_{i,j}^{out}) is 0 or 1.

According to the method of the present invention, by performing causality test on microbial time series data between different detection points, detection points closely related to passenger seats are further selected for subsequent modeling, and spatial dimensions of the detection points are compressed, making the provided data features have strong representation ability.

A specific implementation process of step 4) includes:
I) reading PM_{2.5} concentration, PM₁₀ concentration, CO concentration, NO₂ concentration, SO₂ concentration, and O₃ concentration at the seats, the air supply ports, and the air exhaust ports at P consecutive historical moments, and calculating the total number of bacterial colonies at each detection point at the P consecutive historical moments according to the mapping relationship;
II) reading the total number of bacterial colonies at the i^{th} seat detection point Oᵢ=[Sᵢ^{seat}]ₜ and the total number of bacterial colonies at the air supply port and the air exhaust port which have causality with the i^{th} seat detection point Iᵢ=[Sⱼ^{in/out}, s.t. T_{i,j}^{in/out}=2]ₜ, where Sⱼⁱⁿ is the total number of bacterial colonies at the air supply port, Sⱼ^{out} is the total number of bacterial colonies at the air exhaust port, Sⱼ^{in/out} represents Sⱼⁱⁿ or Sⱼ^{out}, and T_{i,j}^{in/out} represents T_{i,j}ⁱⁿ or T_{i,j}^{out};
III) using Iᵢ as input of a deep echo state network and Oᵢ as output of the deep echo state network, and learning the corresponding relationship between the total number of bacterial colonies at the seat and the total number of bacterial colonies at the air supply port/air exhaust port at different historical moments; and
IV) repeating steps I) to III) for all the seat detection points to obtain the nonlinear description model base of all the seat detection points, where the nonlinear description model base is a set of corresponding relationships of the total number of bacterial colonies at all the seat detection points and the total number of bacterial colonies at the air supply ports/air exhaust ports.

The present invention uses a deep neural network to describe the nonlinear mapping relationship between microorganism and air pollutant concentrations and between microorganism at seat and microorganism at air support port/air exhaust port, thereby ensuring description accuracy.

In step 5), a specific implementation process of calculating fitting results of the total number of bacterial colonies at the air supply ports/air exhaust ports under different ventilation rates includes:
i) increasing the ventilation rate by a fixed value and measuring the total number of bacterial colonies under the corresponding ventilation rate;
ii) performing least square fitting on the total number of bacterial colonies at the k^{th} air supply port/air exhaust port to obtain a polynomial expression of the total number of bacterial colonies Ŝₖ with respect to the ventilation rate vₖ; and
iii) repeating steps i) and ii) for all the air supply ports and all the air exhaust ports, to obtain a polynomial fitting result {Ŝₖ|k=1, 2, 3, . . . , m+n} of the total number of bacterial colonies at all the air supply ports and all the air exhaust ports varying with the ventilation rate, where m and n are numbers of detection points at the air supply ports and the air exhaust ports respectively.

In step 5), an optimization objective is set to simultaneously minimize the fitting result of the total number of bacterial colonies at each seat, and an optimization function is

$$\begin{matrix}min & \{ {\hat{S}}_{1} , {\hat{S}}_{2} , ... , {\hat{S}}_{k} , ... , {\hat{S}}_{m + n} \} \\ s . t . & {\hat{S}}_{k} = g ( {v}_{k} ) , {l}_{k} \leq {v}_{k} \leq {u}_{k}\end{matrix} .$$

The present invention uses a multi-objective optimization method to minimize the total number of bacterial colonies at each seat, so that the degree of microbial contamination in the passenger area reaches overall optimum, and secondary contamination in some areas caused during the adjustment process of the ventilation system is avoided.

In step 5), a non-dominated solution NS*=arg min E, which minimizes an evaluation index

$$E = {\sum }_{k = 1}^{m + n} {\hat{S}}_{k} + Var ( \hat{S} ) ,$$

is selected for determining the ventilation rates NS* of all the air supply ports and all the air exhaust ports, wherein Var(Ŝ) is a variance of the total number of bacterial colonies at all the seats in the test set, and uₖ and lₖ are an upper limit and a lower limit of the ventilation rate vₖ at the k^{th} air supply port/air exhaust port respectively.

The evaluation index is a combination of a cumulative fitting result and the variance of the total number of bacterial colonies at all the seats, where the cumulative fitting result of the total number of bacterial colonies at all the seats represents a degree of microbial contamination after ventilation adjustment and control, and the variance represents a degree of dispersion of microbial contamination among the seats. Selecting the non-dominated solution that minimizes the evaluation index may ensure: (1) the overall degree of microbial contamination in the compartment is minimal; and (2) the difference of microbial contamination among the seats is minimal, so as to avoid extreme contamination in some area/areas.

The present invention further provides a computer apparatus, including a memory, a processor, and a computer program stored on the memory, wherein the processor executes the computer program to implement the steps of the train compartment air adjustment and control method of the present invention.

As an inventive concept, the present invention further provides a computer-readable storage medium, storing a computer program instruction. When the computer program/instruction is executed by a processor, the steps of the train compartment air adjustment and control method of the present invention are implemented.

As an inventive concept, the present invention further provides a computer program product, including a computer program/instruction. When the computer program/instruction is executed by a processor, the steps of the train compartment air adjustment and control method of the present invention are implemented.

Compared with the prior art, the present invention has the following beneficial effects:
1) According to the present invention, microorganisms in a train compartment are tested, analyzed and treated, and a ventilation system adjustment is carried out according to microbial diffusion among detection points, thereby reducing the microbial contamination index in a passenger area. The method has a guide effect on railway train air quality adjustment and control.
2) The present invention studies a mapping relationship between microbial contamination and air pollutant concentration, which can effectively solve a real-time problem of microbial detection and ensure a implementation of real-time adjustment and control on microbial contamination in train compartments.
3) The present invention uses a comprehensive detection manner of multiple detection points at air supply ports, air exhaust ports and seats, which can effectively describe diffusion of air pollutants and microorganisms in the internal environment of compartments and ensure authenticity of depiction of actual spatial distribution by the test results.
4) According to the method of the present invention, causality test is performed on microbial time series data among different detection points, detection points closely related to passenger seats are further selected for subsequent modeling, and spatial dimensions of the detection points are compressed, making the provided data features have strong representation ability.
5) The present invention uses a deep neural network to describe a nonlinear mapping relationship between microorganism and air pollutant concentrations and between microorganisms at seat and microorganisms at air supply port/air exhaust port, thereby ensuring description accuracy.
6) The present invention uses a multi-objective optimization method to minimize the total number of bacterial colonies at each seat, so that the degree of microbial contamination in the passenger area reaches overall optimum, and secondary contamination in some areas caused during the adjustment process of the ventilation system is avoided.

### BRIEF DESCRIPTION OF THE DRAWINGS

FIG. 1 is a flowchart of a method of the present invention.

### DETAILED DESCRIPTION OF THE EMBODIMENTS

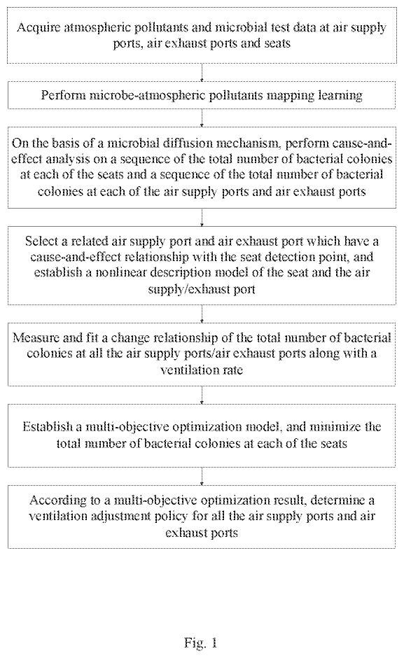

As shown in FIG. 1 a specific implementation process of an embodiment of the present invention is as follows:

Step 1: Collection of Contamination Data at Multiple Detection Points

The interior contamination of a train compartment includes six air pollutants which are PM_{2.5}, PM₁₀, CO, —NO₂, SO₂, and O₃, as well as microbial contamination such as bacteria, fungi, and viruses. Microorganisms are closely related to air quality. Generally, the total number of bacterial colonies in air is positively correlated with probability of existence of pathogenic microorganisms (bacteria, fungi and viruses). Therefore, this patent application measures the pathogenicity of microorganisms by the total number of bacterial colonies as an index. TS WES-C air pollutant detectors (for measuring PM_{2.5} concentration, PM₁₀ concentration, CO concentration, NO₂ concentration, SO₂ concentration, and O₃ concentration in real time) and Anderson impaction air microbial samplers (for measuring the total number of bacterial colonies, which requires 48 h microbial culture) are arranged at multiple air supply ports, air exhaust ports, and seats of the train compartment.

Obtained data includes PM_{2.5} concentration, PM₁₀ concentration, CO concentration, NO₂ concentration, SO₂ concentration, O₃ concentration, and the total number of bacterial colonies at the air supply ports, the air exhaust ports, and the seats, which may be expressed d(i)=[C(i)₁ⁱⁿ, C(i)₂ⁱⁿ, . . . , C(i)ₘⁱⁿ, C(i)₁^{out}, C(i)₂^{out}, . . . , C(i)ₙ^{out}, C(i)₁^{seat}, C(i)₂^{seat}, . . . , C(i)ₚ^{seat}]_{T} and D=[S₁ⁱⁿ, S₂ⁱⁿ, . . . , Sₘⁱⁿ, S₁^{out}, S₂^{out}, . . . , Sₙ^{out}, S₁^{seat}, S₂^{seat}, . . . , Sₚ^{seat}]_{T}, where C(i)ₘⁱⁿ represents the concentration of air pollutants at the m^{th} air supply port, C(i)ₙ^{out} represents the concentration of air pollutants at the n^{th} air exhaust port, C(i)ₚ^{seat} represents the concentration of air pollutants at the p^{th} seat, Sₘⁱⁿ represents the total number of bacterial colonies at the m^{th} air supply port, Sₙ^{out} represents the total number of bacterial colonies at the n^{th} air exhaust port, Sₚ^{seat} represents the total number of bacterial colonies at the p^{th} seat, i represents six air pollutants PM_{2.5}, PM₁₀, CO, NO₂, SO₂, and O₃, and m, n and p are numbers of detection points at the air supply port, the air exhaust port and the seat respectively. Each detection point is regarded as a micro environmental unit, detection data correspond to compartment numbers, time stamps of the detection data are recorded, and an interval between adjacent data is 5 minutes. The collected data is transmitted to a data storage platform in a 4G manner.

Step 2: Learning of Microorganism-Air Pollutant Mapping Relationship

According to historical contamination data of compartment detection points, a model is built to learn a mapping relationship between the total number of bacterial colonies D and the concentration of air pollutants d in each micro environmental unit. A specific modeling process is as follows:
A1: a micro environmental unit is selected, and a data set of air pollutant concentration and total number of bacterial colony indexes of the micro environmental unit in 200 consecutive historical moments is read.
A2: the data set is divided. The foregoing data set includes 200 consecutive historical moments, data of 1-160 moments are used as a training set, and data of 161-200 moments are used as a test set.
A3: a microorganism-air pollutant model is constructed by using a deep belief network, the air pollutant concentration and the total number of bacterial colonies at the same moment are respectively used as input and output of the deep belief network. Layers of the deep belief network are determined by 5-fold cross-validation, and are selected in a range of [1, 2, 3, 4, 5].
A4: the trained deep belief network is tested with the test set, and a group of parameters with highest description accuracy on the test set is selected as a microorganism-air pollutant mapping model of the micro environmental unit.
A5: Steps A1 to A4 are carried out for all micro environmental units (i.e., detection points), to obtain mapping relationships {D=f(d)|i=1, 2, 3, . . . , m+n+p} between the total number of bacterial colonies and the air pollutants of m+n+p detection points, where f represents the mapping relationship.

Step 3: Testing on Causality Among Detection Points Based on Microbial Diffusion Mechanism

Spatial distribution and diffusion of microorganisms in compartments are affected by air movement, and there is causality among the total number of bacterial colonies at the detection points. For each compartment, causality between time series of the total number of bacterial colonies at each seat and each air supply port or air exhaust port is analyzed.

A measured air pollutant concentration data set with a time length of N minutes is selected, the total number of bacterial colonies is calculated according to the mapping relationship obtained in step 2. A time series of the total number of bacterial colonies at the i^{th} seat is denoted as X_{N}ⁱ, a time series of the total number of bacterial colonies at the j^{th} air supply port or air exhaust port is denoted as Y_{N}^{j}, and hypothesis test is performed by using Granger causality test (GCT) to determine whether there is causality between X_{N}ⁱ and Y_{N}^{j} Test result T_{i,j}^{in/out} is output as 0 or 1, wherein 0 represents that there is no causality between the time series X_{N}ⁱ of the total number of bacterial colonies at the seat and the time series Y_{N}^{j} of the total number of bacterial colonies at the air supply port/air exhaust port, while 1 represents that there is causality:

T_{i,j}^{in/out}=GCT(X_{N}^{l}, Y_{N}^{j})

GCT ( ) represents Granger causality test. A test result set of m air supply ports and n air exhaust ports at each seat detection point is obtained:

φᵢ={T_{i,1}ⁱⁿ, T_{i,2}ⁱⁿ, . . . , T_{i,m}ⁱⁿ, T_{i,1}^{out}, T_{i,2}^{out}, . . . , T_{i,n}^{out}}

Step 4: Nonlinear Description of Causality Among Detection Points Modeling

For each seal detection point, a nonlinear description model for an air supply port/air exhaust port related to the seat detection point is built. A specific modeling process is as follows:
B1: PM_{2.5} concentration, PM₁₀ concentration, CO concentration, NO₂ concentration, SO₂ concentration, and O₃ concentration at the seats, the air supply ports, and the air exhaust ports at 100 consecutive historical moments are read, and the total number of bacterial colonies at each detection point at the 100 consecutive historical moments is calculated according to the mapping relationship obtained in step 2.
B2: a data set is divided. The data set includes data at 100 consecutive historical moments, data of 1-60 moments are used as a training set, data of 61-80 moments are used as a validation set, and data of 81-100 moments are used as a test set.
B3: the total number of bacterial colonies at the i^{th} seat detection point Oᵢ=[Sᵢ^{seat}]ₜ and the total number of bacterial colonies at the air supply port and the air exhaust port which have causality with the i^{th} seat detection point Iᵢ=[Sⱼ^{in/out}, s.t. T_{i,j}^{in/out}=1]ₜ are read, where Sⱼⁱⁿ is the total number of bacterial colonies at the air supply port, Sⱼ^{out} is the total number of bacterial colonies at the air exhaust port, Sⱼ^{in/out} represents Sⱼⁱⁿ or Sⱼ^{out}, and T_{i,j}^{in/out} represents T_{i,j}ⁱⁿ or T_{i,j}^{out}.
B4: a nonlinear description model is constructed by using a deep echo state network, with model input Iᵢ and model output Oᵢ, so as to learn a corresponding relationship between the total numbers of bacterial colonies at the seat and the total numbers of bacterial colonies at the air supply port/air exhaust port at different historical moments. The number of reservoir nodes in the deep echo state network is set to 10. The number of reservoir layers and a spectral radius of a reservoir matrix at each layer are determined by 5-fold cross-validation, where the two parameters are selected in ranges of [1, 2, 3, . . . , 10] and [0.1, 0.3, 0.5, 0.7, 0.9] respectively. A trained nonlinear description model h(Iᵢ) is obtained by selecting a group of parameters with highest description accuracy on the validation set.
B5: steps A1 to A4 are carried out for all seat detection points, to obtain a nonlinear description model base {h(Iᵢ)|i=1, 2, 3, . . . , p} of all the seat detection points.

Step 5: Compartment Ventilation Adjustment Strategies Based on Multi-Objective Optimization

C1: a total number of bacterial colonies at all the air supply ports/air exhaust ports changing with ventilation rate is measured according to the following steps:
1) The ventilation rate is increased by a fixed value, the total number of bacterial colonies under the corresponding ventilation rate is measured, and data are recorded in a data storage platform in a format of time stamp-ventilation rate-total number of bacterial colonies.
2) Least square fitting is performed for the k^{th} air supply port/air exhaust port to obtain a polynomial expression of the total number of bacterial colonies Ŝₖ with respect to the ventilation rate vₖ:

Ŝₖ=g(vₖ)
3) The above steps are repeated for all the air supply ports and all the air exhaust ports to obtain a polynomial fitting result {Ŝₖ|k=1, 2, 3, . . . , m+n} of the total number of bacterial colonies at all the air supply ports and all the air exhaust ports changing with the ventilation rate.

C2: A multi-objective optimization model is built. Specific implementation details are as follows:
1) An optimizer is selected and initial super parameters are set: multi-objective grey wolf optimizer is used, and a leader selection mechanism and an archive storage mechanism are embedded to improve convergence ability (MIRJALILI S, SAREMI S, MIRJALILI S M, et al. Multi-objective grey wolf optimizer [J]. Expert Systems With Applications, 2016, 47: 106-19.). A number of search populations, a maximum number of iterations, and an archive size of the multi-objective grey wolf optimizer are set to 200, 100, and 50 respectively.
2) An optimization variable is the ventilation rate at all the air supply ports and all the air exhaust ports, and the search range of the variable satisfies the following formula:

lₖ≤vₖ≤uₖ
uₖ and lₖ are an upper limit and a lower limit of the ventilation rate at the k^{th} air supply port/air exhaust port respectively.
3) According to the polynomial fitting method of the total number of bacterial colonies at the air supply ports and the air exhaust ports changing with the ventilation rate obtained in C1, fitting results of the total number of bacterial colonies at the air supply ports/the air exhaust ports under different ventilation rates are calculated. The total number of bacterial colonies is input to the non-linear description model base of the seat detection points obtained in B5, to output a fitting result of the total number of bacterial colonies at each seat. An optimization objective is set to simultaneously minimize the fitting result of the total number of bacterial colonies at each seat, and an optimization function is:

$$\begin{matrix}min & \{ {\hat{S}}_{1} , {\hat{S}}_{2} , ... , {\hat{S}}_{k} , ... , {\hat{S}}_{m + n} \} \\ s . t . & {\hat{S}}_{k} = g ( {v}_{k} ) , {l}_{k} \leq {v}_{k} \leq {u}_{k}\end{matrix}$$

4) Multi-objective optimization (MIRJALILI S, SAREMI S, MIRJALILI S M, et al. Multi-objective grey wolf optimizer [J]. Expert Systems With Applications, 2016, 47: 106-19.) is performed, and the number of iterations Itr=1 is recorded. Optimization function values of all search results are calculated, and non-dominated solutions are archived.
5) Search paths are updated to generate new ventilation rate search results.
6) The number of searches is It=It+1. If the updated It is less than the maximum number of iterations, return to step 4); otherwise, the multi-objective optimization algorithm ends, and a non-dominated solution set NS in the final archive is output.
7) Performance of the non-dominated solutions on the test set is evaluated, where the evaluation index is a combination of a cumulative fitting result of the total number of bacterial colonies at all the seats and a variance (Var):

$$E = {\sum }_{k = 1}^{m + n} {\hat{S}}_{k} + Var ( \hat{S} )$$

A non-dominated solution NS*=arg min E, which minimizes the evaluation index, is selected for determining the ventilation rates of all the air supply ports and all the air exhaust ports

Step 6: after ventilation adjustment of the train compartments according to the obtained ventilation rate is completed, the total number of bacterial colonies at each detection point is continuously detected, and data are transmitted to the data storage platform.

Step 7: the model does not need to be trained again within a period of time after the first ventilation adjustment is completed, and only calculation is required to be carried out according to the subsequent detection data to output an optimal ventilation adjustment strategy. Because the distribution of microbes in air changes with different crowd behaviors, the causality test, nonlinear description and multi-objective optimization model all require regular training and parameter update to ensure the effectiveness of the model. The retraining time interval may be set to 3 hours.

1. A train compartment air adjustment and control method, wherein comprising the following steps:
1) detecting PM_{2.5} concentration, PM₁₀ concentration, CO concentration, NO₂ concentration, SO₂ concentration, O₃ concentration, and the total number of bacterial colonies at an air supply port, an air exhaust port and a seat of a train;
2) establishing, according to the PM_{2.5} concentration, PM₁₀ concentration, CO concentration, NO₂ concentration, SO₂ concentration, O₃ concentration, and the total number of bacterial colonies at each detection point in a compartment, a mapping relationship between the total number of bacterial colonies D and the concentration of air pollutants d in each micro environmental unit, wherein the micro environmental unit is the detection point;
3) selecting a measured air pollutant concentration data set with a time length of N minutes, calculating the total number of bacterial colonies according to the mapping relationship, denoting a time series of the total number of bacterial colonies at the i^{th} seat as X_{N}ⁱ, denoting a time series of the total number of bacterial colonies at the j^{th} air supply port or air exhaust port as Y_{N}^{j}, performing hypothesis test by using Granger causality test to determine whether there is causality between X_{N}ⁱ and Y_{N}^{j}, and then obtaining a test result set of each seat detection point, m air supply ports and n air exhaust ports;
4) obtaining a nonlinear description model base of all seat detection points according to the mapping relationship and the test result set; and
5) inputting ventilation rates of all air supply ports and all air exhaust ports of the train to a grey wolf optimizer, calculating fitting results of the total number of bacterial colonies at the air supply ports/air exhaust ports under different ventilation rates, inputting the fitting results to the nonlinear description model base to obtain a fitting result of the total number of bacterial colonies at each seat, and determining the ventilation rates of all the air supply ports and all the air exhaust ports by using the fitting result of the total number of bacterial colonies at each seat.

2. The train compartment air adjustment and control method according to claim 1, wherein in step 2), a specific implementation process of establishing a mapping relationship between the total number of bacterial colonies D and the concentration of air pollutants d in each micro environmental unit comprises:
A, reading an index data set of air pollutant concentration and total number of bacterial colonies of the current micro environmental unit at M consecutive historical moments, and dividing the index data set into a training set and a test set;
B, constructing a microorganism-air pollutant model by using a deep belief network, and training the deep belief network by using the air pollutant concentration and the total number of bacterial colonies at the same moment respectively as input and output of the deep belief network;
C, using the test set as input of the trained deep belief network, and selecting a group of parameters with highest description accuracy on the test set as a microorganism-air pollutant mapping model of the micro environmental unit; and
D, repeating steps A-C for all the micro environmental units to obtain the mapping relationship between the total number of bacterial colonies and the air pollutants of m+n+p detection points, where m, n, and p are numbers of detection points at the air supply ports, the air exhaust ports, and the seats respectively.

3. The train compartment air adjustment and control method according to claim 1 wherein in step 3), the test result set is φᵢ={T_{i,1}ⁱⁿ, T_{i,2}ⁱⁿ, . . . , T_{i,m}ⁱⁿ, T_{i,1}^{out}, T_{i,2}^{out}, . . . , T_{i,n}^{out}}, where T_{i,j}ⁱⁿ is a test result of the air supply port, T_{i,j}ⁱⁿ=GCT(X_{N}ⁱ, Y_{N}^{j}), T_{i,j}^{out} is a test result of the air exhaust port, and T_{i,j}^{out}=GCT(X_{N}ⁱ, Y_{N}^{j}); value of the test result T_{i,j}ⁱⁿ is 0 or 1, and value of the test result T_{i,j}^{out} is 0 or 1; and GCT ( ) represents Granger causality test.

4. The train compartment air adjustment and control method according to claim 3, wherein a specific implementation process of step 4) comprises:
I) reading PM_{2.5} concentration, PM₁₀ concentration, CO concentration, NO₂ concentration, SO₂ concentration, and O₃ concentration at the seats, the air supply ports, and the air exhaust ports at P consecutive historical moments, and calculating the total number of bacterial colonies at each detection point at the P consecutive historical moments according to the mapping relationship;
II) reading the total number of bacterial colonies at the i^{th} seat detection point Oᵢ[Sᵢ^{seat}]ₜ and the total number of bacterial colonies at the air supply port and the air exhaust port which have causality with the i^{th} seat detection point Iᵢ=[Sⱼ^{in/out}, s.t. T_{i,j}^{in/out}=1]ₜ, where Sⱼⁱⁿ is the total number of bacterial colonies at the air supply port, Sⱼ^{out} is the total number of bacterial colonies at the air exhaust port, Sⱼ^{in/out} represents Sⱼⁱⁿ or Sⱼ^{out}, and T_{i,j}^{in/out} represents T_{i,j}ⁱⁿ or T_{i,j}^{out};
III) using Iᵢ as input of a deep echo state network and Oᵢ as output of the deep echo state network, and learning the corresponding relationship between the total number of bacterial colonies at the seat and the total number of bacterial colonies at the air supply port/air exhaust port in different historical moments; and
IV) repeating steps I) to III) for all the seat detection points to obtain the nonlinear description model base of all the seat detection points, where the nonlinear description model base is a set of corresponding relationships of the total number of bacterial colonies at all the seat detection points and the total number of bacterial colonies at the air supply ports/air exhaust ports.

5. The train compartment air adjustment and control method according to claim 2, wherein in step 5), a specific implementation process of calculating fitting results of the total number of bacterial colonies at the air supply ports/air exhaust ports under different ventilation rates comprises:
i) increasing the ventilation rate by a fixed value and measuring the total number of bacterial colonies under the corresponding ventilation rate;
ii) performing least square fitting on the total number of bacterial colonies at the k^{th} air supply port/air exhaust port to obtain a polynomial expression g(vk) of the total number of bacterial colonies Ŝₖ with respect to the ventilation rate vₖ; and
iii) repeating steps i) and ii) for all the air supply ports and all the air exhaust ports, to obtain a polynomial fitting result {Ŝₖ|k=1,2,3, . . . , m+n} of the total number of bacterial colonies at all the air supply ports and all the air exhaust ports changing with the ventilation rate, where m and n are numbers of detection points at the air supply ports and the air exhaust ports respectively.

6. The train compartment air adjustment and control method according to claim 5, wherein in step 5), an optimization objective is set to simultaneously minimize the fitting result of the total number of bacterial colonies at each seat, and an optimization function is
$$\begin{matrix}min & \{ {\hat{S}}_{1} , {\hat{S}}_{2} , ... , {\hat{S}}_{k} , ... , {\hat{S}}_{m + n} \} \\ s . t . & {\hat{S}}_{k} = g ( {v}_{k} ) , {l}_{k} \leq {v}_{k} \leq {u}_{k}\end{matrix} ,$$
where uₖ and lₖ are respectively an upper limit and a lower limit of the ventilation rate at the k^{th} air supply port/air exhaust port.

7. The train compartment air adjustment and control method according to claim 6, wherein in step 5), a non-dominated solution NS*=arg min E, which minimizes an evaluation index E=Σₖ₌₁^{m+n}Ŝₖ+Var(Ŝ), is selected for determining the ventilation rates NS* of all the air supply ports and all the air exhaust ports, where Var(Ŝ) is a variance of the total number of bacterial colonies at all the seats in the test set.

8. A computer apparatus, comprising a memory, a processor, and a computer program stored on the memory, wherein the processor executes the computer program to implement the steps of the method according to claim 1.

9. A computer-readable storage medium, storing a computer program/instruction, wherein when the computer program/instruction is executed by a processor, the steps of the method according to claim 1 are implemented.

10. A computer program product, comprising a computer program/instruction, wherein when the computer program/instruction is executed by a processor, the steps of the method according to claim 1 are implemented.
